(19) United States
(12) Patent Application Publication
(10) Pub. No.: US 2015/0328427 A1
(43) Pub. Date: Nov. 19, 2015

(54) PULSE-WIDTH MODULATION METHOD FOR CONTROLLING OXYGEN CONCENTRATION IN ANESTHETIC MACHINE OR VENTILATOR

(71) Applicant: BEIJING AEONMED CO., LTD., Beijing (CN)

(72) Inventor: Jie CHENG, Beijing (CN)

(21) Appl. No.: 14/397,083

(22) PCT Filed: Oct. 22, 2013
(86) PCT No.: PCT/CN2013/085681

§ 371 Date: Oct. 24, 2014

(30) Foreign Application Priority Data
Dec. 26, 2012 (CN) 201210575443.4

(51) Int. Cl. A61M 16/10; A61M 16/20; A61M 16/00

(52) U.S. Cl. CPC A61M 16/1005; A61M 16/104; A61M 16/0069; A61M 16/201; A61M 2202/0208; A61M 2202/0241; A61M 2205/3334

(57) ABSTRACT

A pulse-width modulation method for controlling oxygen concentration in an anesthetic machine or in a ventilator, comprising the following steps: step A, with a predetermined time interval as one pulse interval, a processing unit divides a breathing cycle into multiple consecutive pulse intervals; step B, a data calculation unit calculates the average inspiratory flow in a certain time interval on the basis of inspiratory flows in one cycle that are detected by a detecting unit, and then calculates the average oxygen flow of this stage on the basis of the average inspiratory flow; and, step C, a control unit selects a solenoid valve and controls the opening and closing times thereof on the basis of the average oxygen flow as calculated in the previous step to implement control of the oxygen flow for each interval.

### FIELD OF THE INVENTION

The present invention relates to the field of control technologies for an oxygen gas concentration in a ventilator and an anesthetic machine, and in particular, to a pulse-width modulated method for controlling an oxygen concentration in an anesthetic machine or a ventilator.

### BACKGROUND OF THE INVENTION

The oxygen concentration in the gas delivered from a ventilator is an essential index of the ventilation to a patient. In an existing air-oxygen mixed ventilator as clinically employed, the oxygen concentration is adjustable in a range of 21%-100%. In order to eliminate the anoxic status of a patient as soon as possible, as required by therapy, the oxygen concentration needs to be adjusted to a certain level by adjusting compressed air and high-pressure oxygen gas according to a proportion set by the ventilator, thus if one of the compressed air and the high-pressure oxygen gas is blocked, only the other one of the compressed air and the high-pressure oxygen gas can be output. For example, if the oxygen gas is blocked, only the air can be output; and if the air is blocked, only the 100% oxygen gas can be output, and hence the patient will be poisoned to death due to the inhalation of a large amount of pure oxygen for a long time.

As can be seen from the above analysis, during the ventilation by a ventilator to a patient, the oxygen gas concentration needs to be precisely controlled. In the prior art, in order to improve the precision in adjusting the oxygen gas concentration, a proportional valve is adopted in some ventilators. Although the proportional valve can somewhat improve the precision in controlling the oxygen gas concentration, the cost is much increased, thus the application of the proportional valve is limited. In another common implementation, a needle valve is used for controlling the oxygen concentration for example in Chinese Patent Application No. 01261459.9, where a screw flow regulating valve for a ventilator is provided based on a principle that direct-proportional adjustment of the oxygen flow or oxygen concentration is realized by adjusting the opening degree (0-120°) of a valve needle of a needle valve. However, the needle valve is manufactured by a pure mechanical technique, thus the adjustment error range of the needle valve is directly affected by the machining precision of the mechanical technique, and even among needle valves of the same batch, the error range of the parameter adjustment is significant; further, the installation process of the needle valve is relatively complex, thus the production cost will be increased, and the requirement of the current ventilator market cannot be met.

### SUMMARY OF THE INVENTION

Therefore, it is an object of the present invention to provide a pulse-width modulated method for controlling an oxygen concentration in an anesthetic machine or a ventilator, to precisely control the oxygen gas concentration, so that the oxygen supply of a ventilator safe, stable and reliable.

To attain the above object, the invention employs the following technical solutions.

A pulse-width modulated method for controlling an oxygen concentration in an anesthetic machine or a ventilator includes:

Step A: dividing, by a processing unit, a respiratory cycle into a plurality of consecutive pulse intervals by taking a predetermined time interval as a pulse interval;

Step B: calculating, by a data computing unit, an average inspiratory flow quantity in each pulse interval according to an inspiratory flow quantity detected by a detection unit in a respiratory cycle, and then calculating an average oxygen flow quantity in the pulse interval according to the average inspiratory flow quantity; and

Step C: selecting, by a control unit, an appropriate electromagnetic valve according to the average oxygen flow quantity in each pulse interval calculated in Step B, and controlling, by the control unit, an opening time and a closed time of the electromagnetic valve, thereby controlling the oxygen flow quantity in the pulse interval.

As a preferred embodiment of the above pulse-width modulated method for controlling an oxygen concentration in an anesthetic machine or a ventilator, the average inspiratory flow quantity in each pulse interval in Step B is an average inspiratory flow quantity of a corresponding pulse interval in an immediately previous respiratory cycle.

As a preferred embodiment of the above pulse-width modulated method for controlling an oxygen concentration in an anesthetic machine or a ventilator, in Step C, the oxygen flow quantity is controlled by the opening and closing of at least one electromagnetic valve.

As a preferred embodiment of the above pulse-width modulated method for controlling an oxygen concentration in an anesthetic machine or a ventilator, a sum of flow throughputs of all the at least one electromagnetic valve is greater than 120 litre/minute.

As a preferred embodiment of the above pulse-width modulated method for controlling an oxygen concentration in an anesthetic machine or a ventilator, the control unit controls the opening and closing of one or more electromagnetic valves according to the oxygen flow quantity, and the electromagnetic valves are selected for controlling based on a principle of preferentially using a small-flow valve and a principle of avoiding opening a big-flow valve momentarily as possible.

As a preferred embodiment of the above pulse-width modulated method for controlling an oxygen concentration in an anesthetic machine or a ventilator, the opening time and the closed time of an electromagnetic valve in the pulse interval are determined by a duty ratio of the electromagnetic valve.

As a preferred embodiment of the above pulse-width modulated method for controlling an oxygen concentration in an anesthetic machine or a ventilator, duration of the pulse interval in Step A is constant.

As a preferred embodiment of the above pulse-width modulated method for controlling an oxygen concentration in an anesthetic machine or a ventilator, the average inspiratory flow quantity in the pulse interval in Step B is calculated by a formula:

$${\overline{Q}}_{{dT}_{i}} = \frac{{\int }_{{t}_{i}}^{{t}_{i} + {dT}_{i}} {Q}_{i} \cdot d t}{{dT}_{i}} ,$$

where, Q̅_{dT}_{ᵢ} represents the start time of the pulse interval, represents the average inspiratory flow quantity in the pulse interval, and a represents an instantaneous flow quantity.

As a preferred embodiment of the above pulse-width modulated method for controlling an oxygen concentration in an anesthetic machine or a ventilator, the average oxygen flow quantity is calculated as Q̅_{O2dT}_{ᵢ}= Q̅_{dT}_{ᵢ}(FiO2−21), where, Q_{O2} represents the oxygen flow quantity, a represents the inspiratory flow quantity, and FiO2 represents a set value of the oxygen concentration.

As a preferred embodiment of the above pulse-width modulated method for controlling an oxygen concentration in an anesthetic machine or a ventilator, the duration of the pulse interval is 200 ms.

The beneficial effects of the invention are described below. With the pulse-width modulated method for controlling an oxygen concentration in an anesthetic machine or a ventilator provided in the invention, a respiratory cycle is divided into a plurality of consecutive stages, i.e. equally spaced pulse cycles, the oxygen flow quantity in each stage is calculated, and then the oxygen flow quantity in each stage is controlled by controlling the opening and closing of an electromagnetic valve, thereby the oxygen gas concentration during ventilation can be controlled precisely, and the safeness and stability of the ventilator or the anesthetic machine is improved.

### BRIEF DESCRIPTION OF THE DRAWINGS

FIG. 1 is a graph of an inspiratory flow quantity according to an embodiment of the invention;

FIG. 2 is a graph of an ideal oxygen flow quantity according to an embodiment of the invention; and

FIG. 3 is diagram showing an approximate oxygen flow quantity according to an embodiment of the invention.

### DETAILED DESCRIPTION OF THE EMBODIMENTS

The technical solutions of the invention will be further illustrated below in conjunction with the accompanying drawings and specific embodiments.

The invention provides a pulse-width modulated method for controlling an oxygen concentration in an anesthetic machine or a ventilator, and the method may be used to precisely control the oxygen concentration during ventilation of a ventilator. The basis for realizing the method lies in that: in an inspiration time, the flow quantity of oxygen gas may be calculated from a set value of the tidal volume and a set value of the oxygen concentration, and it is assumed that the flow rate of the oxygen gas in each of a plurality of stages divided from the inspiration time is variable, where the inspiration time is divided into the plurality of stages for example in a unit of 200 milliseconds (ms), and then the oxygen flow quantity in each stage of 200 ms is controlled, and the closing and opening of an electromagnetic valve is controlled according to the oxygen flow quantity calculated in the stage, so that a precise oxygen concentration can be obtained. The method specifically includes:

Step A: dividing, by a processing unit, a respiratory cycle into a plurality of consecutive pulse intervals by taking a predetermined time interval as the pulse interval;

Step B: calculating, by a data computing unit, an average inspiratory flow quantity in a certain pulse interval according to an inspiratory flow quantity detected by a detection unit in a respiratory cycle, and then calculating an average oxygen flow quantity in this pulse interval according to the calculated average inspiratory flow quantity; and

Step C: selecting, by a control unit, an appropriate electromagnetic valve according to the average oxygen flow quantity in each pulse interval calculated in Step B, and controlling, by the control unit, an opening time and a closed time of the electromagnetic valve, thereby controlling the oxygen flow quantity in the pulse interval.

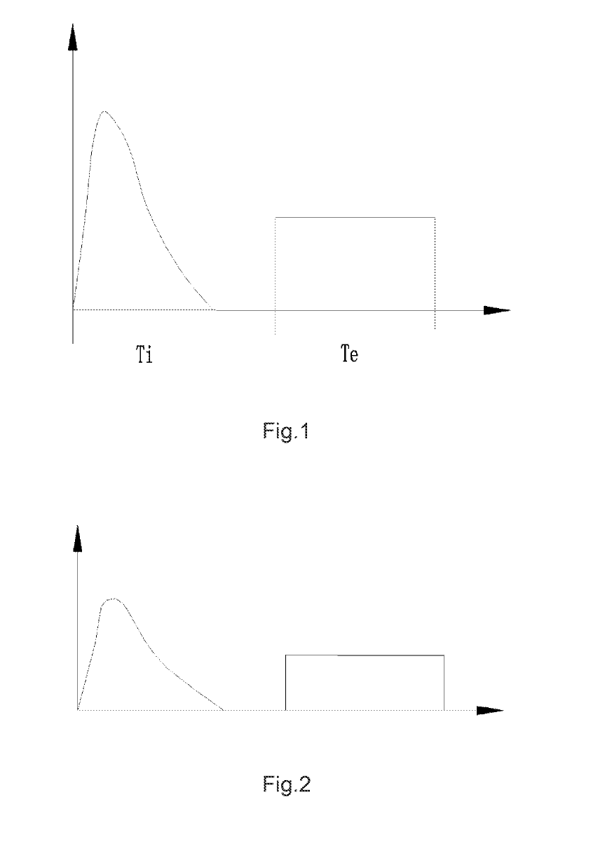

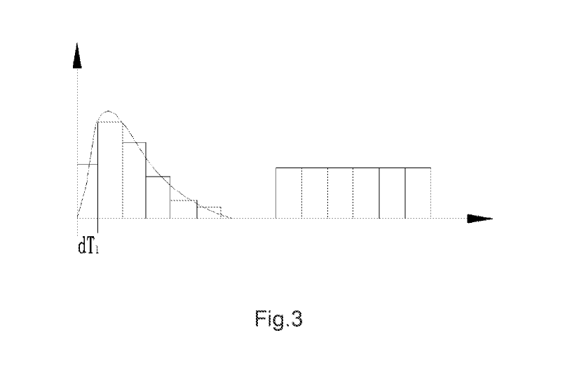

As shown in FIG. 1 to FIG. 3, it is assumed in Step A that the oxygen gas flow rate varies with time, and the respiratory cycle is divided into a plurality of consecutive intervals. Here, the division of the respiratory cycle into the pulse intervals is to determine each pulse interval dT, and a principle for the division is to make dT as a constant value if possible. In this application, the pulse interval has duration of 200 ms, but it is not limited to 200 ms. In practice, the duration of the pulse interval may be selected as required, and hence may be lengthened or shortened appropriately in certain cases (especially in the case of the last pulse interval of inspiration time or expiration time); for example, in the case of inspiration time Ti=1250 ms and expiration time Te=1501 ms, the pulse intervals dT after the division are as follows:

{[200,200,200,200,200,250]; [200,200,200,200,200,200,200,101]}.

Because in most modes (except for a completely-instructed ventilation mode) of the anesthetic machine or ventilator, the inspiration time and the expiration time are not predefined values, the pulse intervals dT are automatically generated in the immediate previous respiratory cycle (except for synchronized intermittent mandatory ventilation (SIMV)), and the determination of the length of the last pulse interval of inspiration or expiration is critical.

The data computing unit calculates the average inspiratory flow quantity in a certain pulse interval according to an inspiratory flow quantity detected by a detection unit in a respiratory cycle, and then calculates the average oxygen flow quantity in this pulse interval according to the calculated average inspiratory flow quantity. Here, the average inspiratory flow quantity refers to an average inspiratory flow quantity in one pulse interval, that is, the average inspiratory flow quantity of the corresponding pulse interval starting from time tᵢ in the immediately previous respiratory cycle, and may be calculated by formula 1:

$${\overline{Q}}_{{dT}_{i}} = \frac{{\int }_{{t}_{i}}^{{t}_{i} + {dT}_{i}} {Q}_{i} \cdot d t}{{dT}_{i}} ,$$

where, tᵢ represents the start time of a certain pulse interval, Q̅_{dT}_{ᵢ} represents the average inspiratory flow quantity in this pulse interval, and Qᵢ represents an instantaneous flow quantity. This formula 1 means in nature that the average inspiratory flow quantity equals to an integral of instantaneous flow quantities with time.

Ideally, as shown in FIG. 1 and FIG. 2, the oxygen flow quantity should strictly meet formula 2 below:

Q_{O2}=Qᵢ(FiO2−21)/79,

where, Q_{O2} represents the oxygen flow quantity, Qᵢ represents the inspiratory flow quantity, and FiO2 represents a set value of the oxygen concentration.

According to formulas 1 and 2, a formula 3 of the average oxygen flow quantity Q̅_{O2dT}_{ᵢ} corresponding to a certain average inspiratory flow quantity may be obtained as:

Q̅_{O2dT}_{ᵢ}= Q̅_{dT}_{ᵢ}(FiO2−21)/79.

Corresponding to the above example of division of the pulse intervals, the oxygen flow quantities in the pulse intervals may be calculated according to the above formulas 1, 2 and 3 as:

{[4.5, 9, 6, 3.5, 1.2, 0]; [7.9, 7.3, 7.3, 7.3, 7.3, 7.3, 7.3, 7.3]}.

According to the average oxygen flow quantity calculated according to formula 3, the control unit controls the closing and opening of an electromagnetic valve to control the average oxygen flow quantity; in order to improve the precision in controlling the oxygen flow quantity and flow rate, the flow quantity of oxygen gas is controlled in Step C by the opening and closing of at least one electromagnetic valve. Because 120 litre/minute is a required performance index of a machine, that is, the maximum flow rate provided by the machine cannot be lower than 120 litre/minute, a sum of the flow throughputs of all the electromagnetic valves should be greater than 120 litre/minute.

The time for the opening and closing of the at least one electromagnetic valve is designated once in each pulse interval. Because there may be various schemes for approximately achieving a certain oxygen flow quantity, two principles, i.e. (1) a principle of using small-flow valves preferentially and (2) a principle of avoiding opening of big-flow valves momentarily if possible, are employed. Herein, the first principle 1) is used for determining which valves may be opened at most in a certain pulse interval; and the second principle (2) is used for selecting certain valve(s) to be opened from the valves determined by the first principle (1).

A duty ratio refers to a proportion between the opening time and closed time of a valve in a certain pulse interval, and is essential in this application. In a pulse interval dT, the state of each valve may be represented by Tvi=[To,Tc], where To represents the opening time of the valve, and Tc represents the closed time of the valve. If a valve is closed, To=0 and Tc=dT, and so on. A group of valve states may be determined for each pulse interval, for example, for 8 valves, TV={Tv1, Tv2, Tv3, Tv4, Tv5, Tv6, Tv7, Tv8}.

The oxygen flow quantity in each pulse interval is calculated by formula 3, the optimum valve is found, and the opening and closing of the electromagnetic valve is controlled in a time period of 200 ms, thereby the oxygen concentration may be controlled precisely. Now, the control method of the invention is further illustrated by an example: for example, in the case of 8 electromagnetic valves (but the number of electromagnetic valves is not limited to 8 in the invention, and the larger the number of the electromagnetic valve is, the more precisely the oxygen concentration will be controlled), if the flow throughputs of the 8 electromagnetic valves are respectively Qv=[4, 4, 12, 12, 30, 30, 30, 30] and the oxygen flow quantity to be expected from the control in a certain pulse interval is determined by formula 3 as 9, then by searching among the valves in an order of increasing flow throughputs according to the first principle, it may be found that three valves with the respective flow throughputs of 4, 4 and 12, i.e. the first, second and third valves, might be used at most, and then it will be found by searching among these 3 valves in an order of decreasing flow throughputs according to the second principle that only the valve with a flow throughput of 12 (i.e. the third valve) needs to be opened, and the state of the third valve is [9*200/12, 3*200/12]. As such, the oxygen concentration is controlled by the opening and closing of the electromagnetic valve.

The technical principles of the invention have been described above in conjunction with specific embodiments. These descriptions are only used for explaining the principles of the invention, rather than limiting the protection scope of the invention in any way. Other specific implementations may be made by one skilled in the art based on the explanation herein without creative work, and all these implementations will fall into the protection scope of the invention.

1. A pulse-width modulated method for controlling an oxygen concentration in an anesthetic machine or a ventilator, comprising:
Step A: dividing, by a processing unit, a respiratory cycle into a plurality of consecutive pulse intervals by taking a predetermined time interval as a pulse interval;
Step B: calculating, by a data computing unit, an average inspiratory flow quantity in each pulse interval according to an inspiratory flow quantity detected by a detection unit in a respiratory cycle, and then calculating an average oxygen flow quantity in the pulse interval according to the average inspiratory flow quantity; and
Step C: selecting, by a control unit, an appropriate electromagnetic valve according to the average oxygen flow quantity in each pulse interval calculated in Step B, and controlling, by the control unit, an opening time and a closed time of the electromagnetic valve, thereby controlling the oxygen flow quantity in the pulse interval.

2. The pulse-width modulated method for controlling an oxygen concentration in an anesthetic machine or a ventilator according to claim 1, wherein, the average inspiratory flow quantity in each pulse interval in Step B is an average inspiratory flow quantity of a corresponding pulse interval in an immediately previous respiratory cycle.

3. The pulse-width modulated method for controlling an oxygen concentration in an anesthetic machine or a ventilator according to claim 1, wherein, in Step C, the oxygen flow quantity is controlled by the opening and closing of at least one electromagnetic valve.

4. The pulse-width modulated method for controlling an oxygen concentration in an anesthetic machine or a ventilator according to claim 3, wherein, a sum of flow throughputs of all the at least one electromagnetic valve is greater than 120 litre/minute.

5. The pulse-width modulated method for controlling an oxygen concentration in an anesthetic machine or a ventilator according to claim 3, wherein, the control unit controls the opening and closing of one or more electromagnetic valves according to the oxygen flow quantity, and the electromagnetic valves are selected for controlling based on a principle of preferentially using a small-flow valve and a principle of avoiding opening a big-flow valve momentarily as possible.

6. The pulse-width modulated method for controlling an oxygen concentration in an anesthetic machine or a ventilator according to claim 3, wherein, the opening time and the closed time of an electromagnetic valve in the pulse interval are determined by a duty ratio of the electromagnetic valve.

7. The pulse-width modulated method for controlling an oxygen concentration in an anesthetic machine or a ventilator according to claim 1, wherein, duration of the pulse interval in Step A is constant.

8. The pulse-width modulated method for controlling an oxygen concentration in an anesthetic machine or a ventilator according to claim 2, wherein, the average inspiratory flow quantity in the pulse interval in Step B is calculated by a formula:
$${\overline{Q}}_{{dT}_{i}} = \frac{{\int }_{{t}_{i}}^{{t}_{i} + {dT}_{i}} {Q}_{i} \cdot d t}{{dT}_{i}}$$
wherein, tᵢ represents the start time of the pulse interval, Q̅_{dT}_{ᵢ} represents the average inspiratory flow quantity in the pulse interval, and Qᵢ represents an instantaneous flow quantity.

9. The pulse-width modulated method for controlling an oxygen concentration in an anesthetic machine or a ventilator according to claim 8, wherein, the average oxygen flow quantity is calculated as Q̅_{O2dT}_{ᵢ}= Q̅_{dT}_{ᵢ}(FiO2<21)/79, wherein, FiO2 represents a set value of the oxygen concentration.

10. The pulse-width modulated method for controlling an oxygen concentration in an anesthetic machine or a ventilator according to claim 9, wherein, duration of the pulse interval is 200 ms.
